(19) United States
(12) Patent Application Publication
(10) Pub. No.: US 2017/0296713 A1
(43) Pub. Date: Oct. 19, 2017

(54) ATRAUMATIC SELECTIVE SURGICAL SUCTION DEVICE AND METHOD

(71) Applicant: Indiana University Research and Technology Corporation, Indianapolis, IN (US)

(72) Inventor: Brian C. Lobo, Indianapolis, IN (US)

(73) Assignee: Indiana University Research and Technology Corporation, Indianapolis, IN (US)

(21) Appl. No.: 15/510,153

(22) PCT Filed: Sep. 11, 2015
(86) PCT No.: PCT/US2015/049630

§ 371 Date: Mar. 9, 2017

Related U.S. Application Data
(60) Provisional application No. 62/049,626, filed on Sep. 12, 2014.

(51) Int. Cl. A61M 1/00

(52) U.S. Cl. CPC A61M 1/008; A61M 1/0033; A61M 1/0066

(57) ABSTRACT

Suction devices are disclosed comprising a side wall defining a lumen, an open proximal end sized to provide coupling to a suction source, a distal wall opposite the open proximal end, the distal wall at least partially occluding the lumen, the side wall extending between the proximal end and the distal wall, a plurality of vent holes in communication with the lumen defined in the side wall, and at least one vent hole in communication with the lumen defined in the distal wall. Also disclosed are methods and additional suction devices.

### CROSS-REFERENCE TO RELATED APPLICATIONS

This application claims priority to U.S. Provisional Application Ser. No. 62/049,626, filed on Sep. 12, 2014, the entire disclosure of which is hereby expressly incorporated by reference.

### FIELD OF THE DISCLOSURE

The present disclosure is related to methods and devices to provide suction during a surgical procedure. The disclosure is more particularly directed to methods and devices that provide surgical suction in a manner that reduces a likelihood of tissue trauma resulting therefrom.

### BACKGROUND

Suction devices are essential for many traditional surgical approaches. Functionally, a surgical suction rapidly removes unwanted debris from the surgical field. The utility of surgical suction is at least partially dependent upon being able to continuously operate without clogging, being user friendly, being ergonomic, and being compatible with existing operating room vacuum systems and tubing. Many modern surgical approaches place additional demands on surgical suction devices, namely the ability to suction fluids and semisolids without aspirating adjacent tissue or vital structures. That is, a selective or atraumatic suction device. These abilities are particularly useful for suctioning around cranial nerves, arteries, brain parenchyma, orbital contents and other vital structures; for placement of grafts and microflaps; and for use anywhere that delicate tissue handling with fluid evacuation is needed.

Various devices have attempted to fill this void-Rhoton, Brackmann, McCulloch suctions utilizing tip displacement or partial fenestrations; Poole suctions utilizing a two piece construction with a fenestrated guard; microsuction cannulas created to be so small that they are incapable of suctioning the adjacent tissue; atraumatic suction tips with bulbous or teardrop shaped tips that do not score or lacerate tissue. More elaborate systems such as those made by NICO also allow modulation of suction pressure, but not without significant cost and increased operator demand, and ultimately loss of suction. The existing devices typically provide effective suction or delicate tissue handling capabilities but not both. Accordingly, what is needed is a device that provides effective suction and delicate tissue handling capabilities.

### SUMMARY

According to a first embodiment, the present disclosure includes a suction device including: a cylindrical wall defining a lumen; an open proximal end sized to provide coupling to a suction source; a planar distal wall opposite the open proximal end, the planar distal wall at least partially occluding the lumen, the cylindrical wall extending between the proximal end and the distal wall; a plurality of vent holes in communication with the lumen defined in the cylindrical wall; and at least one vent hole in communication with the lumen defined in the planar distal wall.

According to another embodiment of the present disclosure, a suction device is provided including: a tube-shaped body having a lumen formed therethrough, an outer surface, a distal tip defining a planar surface having at least two openings therein, the at least two openings being in communication with the lumen, a proximal end having an opening in communication with the lumen, the proximal end shaped to couple to a suction source, and a distal end having a planar distal wall, the planar distal wall having at least one opening in communication with the lumen, the at least one opening having a diameter smaller than a diameter of the lumen.

According to another embodiment of the present disclosure, a method is provided that includes: obtaining a suction device including: a cylindrical wall defining a lumen; an open proximal end sized to provide coupling to a suction source; a planar distal wall opposite the open proximal end, the planar distal wall at least partially occluding the lumen, the cylindrical wall extending between the proximal end and the distal wall; a plurality of vent holes in communication with the lumen defined in the cylindrical wall; and at least one vent hole in communication with the lumen defined in the planar distal wall; placing the proximal end of the suction device in communication with a suction source; engaging matter with the planar distal wall such that said matter adheres to the planar distal wall via suction supplied by the suction source without allowing said matter to enter the lumen; placing the matter at a desired location; and causing a reduction in suction experienced at the planar distal wall to allow disengagement of the matter from the planar distal wall.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, wherein:

FIG. 1 is a perspective view of a first embodiment of a device of the present disclosure;

FIG. 2 is an end view of the device of FIG. 1;

FIG. 3 is a cross sectional view of the device of FIG. 1; and

FIG. 4 is a perspective view of a suction wand usable with the device of FIG. 1.

### DETAILED DESCRIPTION OF EMBODIMENTS

The embodiments disclosed herein are not intended to be exhaustive or limit the invention to the precise form disclosed in the following detailed description. Rather, the embodiments were chosen and described so that others skilled in the art may utilize their teachings.

FIG. 1 shows an exemplary suction tip 10. Suction tip 10 is shown as a hollow cylinder. Suction tip 10 is illustratively constructed from plastic. However, embodiments are envisioned where suction tip 10 is constructed from other materials, such as metal, particularly biocompatible metals of the types often used for surgical instrumentation. Suction tip 10 is discussed herein as a device this is separate from a suction wand 400 with which it is used. However, it should be appreciated that embodiments are envisioned where the tip 10 is integral with suction wand 400 or catheter such that the described features attributed to tip 10 are attributed to suction wand 400 or a suction catheter.

Suction tip 10 includes a proximal end 12 and distal end 14. A cylindrical wall 16 extends between proximal end 12 and distal end 14. Proximal end 12 is illustratively open such that no proximal wall exists except for that provided by the wall thickness of cylindrical wall 16. Distal end 14 includes distal wall 18.

Cylindrical wall 16 and distal wall 18 are illustratively of the same thickness. However, embodiments are envisioned where the walls 16, 18 have differing thicknesses. Furthermore, in at least one embodiment, cylindrical wall 16 itself does not have a constant thickness. In one embodiment, a change in thickness of cylindrical wall 16 is provided at point 20 near the distal end 14 of tip 10. This change in thickness can be temporary (spatially) so as to approximate an internal ring or can be permanent such that all points distal to the point of change assume the same (or greater) thickness. The change in thickness at point 20 causes a reduction of the internal diameter of tip 10.

The internal diameter of the proximal end 12 of tip 10 is chosen to snugly fit over an associated suction wand 400. Suction wand 400 is received in tip 10 at proximal end 12. The change in thickness at point 20 limits the depth to which suction wand 400 may extend within distal end 14. It should be appreciated that suction wands 400 come in many different sizes. These sizes are often referred to by their outer diameter (measured in French) which is substantially uniform (varying only in manufacturing tolerances).

In one embodiment, the cylindrical wall 16 of the distal end 14 (more distal than point 20), includes a plurality of vent holes 30. In the illustrated embodiment, there are four sets of three vent holes 30. Within each set, the three vent holes 30 are axially aligned. Each of the four sets are offset by 90-degrees on the circle defined by the cylindrical wall 16. Embodiments are envisioned where a differing number of vent holes are used and where a different number of sets are used. Still further, embodiments are envisioned where vent holes 30 are differently aligned with other vent holes 30. In one embodiment, the most-distal circumferential vent holes 30 are spaced apart from the distal end 14 of tip 10 by a distance equal to one-half of the radius of the vent hole 30 plus the thickness of distal wall 18.

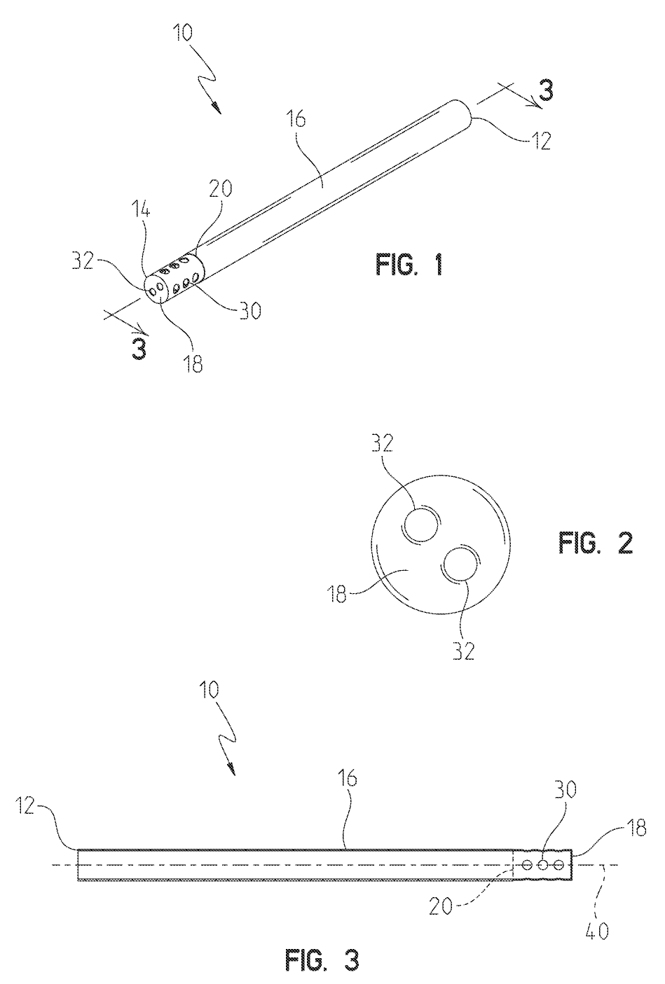

Distal wall 18 is illustratively planar and includes one or more vent holes 32 defined therein. The embodiment shown in FIGS. 1-3 includes two vent holes 32 in the distal wall 18. The two vent holes 32 are illustratively aligned such that an axis that connects them is equidistant from the sets of vent holes 30. Stated differently, while the shown vent holes 30 in the cylindrical wall 16 are oriented at 0, 90, 180, and 270-degrees, the axis connecting vent holes 32 is oriented at 45 and 225-degrees. Of course, this orientation is illustrative only. As shown in FIG. 3, distal wall 18 is perpendicular to a longitudinal axis 40 of tip 10. However, embodiments are envisioned where distal wall 18 is not perpendicular to longitudinal axis 40.

Operationally, the device of the present disclosure is used in surgical applications. Suction, via devices such as suction wand 400 allow for evacuation of material and liquids from a surgical field. Such suction is often a product of a connection to vacuum ports typically available in a surgical suite and/or in the hospital setting generally. The strength of the vacuum being pulled and/or the suction being provided at the distal tip of suction wand 400 is not typically customizable other than through the occlusion or non-occlusion of a vent hole in the handle of suction wand 400. The occlusion or non-occlusion of the vent hole in the handle is more akin to an on/off setting than the selection of a particular suction strength. Accordingly, while suction wand 400 is able to clear items from the surgical field, suction wand 400 is less suited for the careful manipulation of delicate material, or use around sensitive tissues that are potentially adversely affected by intense suction.

It should be appreciated that upon placement of tip 10 on wand 400, the output suction provided by wand 400 is distributed over all vent holes 30, 32. The suction also serves to pull tip 10 onto wand 400 and to hold it there. However, embodiments are envisioned where a detent in cylindrical wall 16 or some other device for holding tip 10 on wand 400 is provided.

The illustrated embodiment has twelve circumferential vent holes 30 and two end vent holes 32. Embodiments are envisioned where the circumferential vent holes 30 define a diameter that is between 15-45% of the interior diameter of the tip 10. More particularly, embodiments are envisioned where circumferential vent holes 30 define a diameter that is between 30-35% of the interior diameter of the tip 10. Similarly, embodiments are envisioned where the end (tip) vent holes 32 define a diameter that is between 10-30% of the interior diameter of the tip 10. More particularly, embodiments are envisioned where end/tip vent holes 32 define a diameter that is between 15-25% of the interior diameter of the tip 10. Further, embodiments are envisioned where the end/tip vent holes 32 define a diameter that is between 50-70% of the diameter of circumferential vent holes 30. More particularly, embodiments are envisioned where the end/tip vent holes 32 define a diameter that is between 55-65% of the diameter of circumferential vent holes 30.

Still further higher flow embodiments are envisioned where the end/tip vent holes 32 define a diameter that is between 80-100% of the diameter of the circumferential vent holes 30 and the end/tip vent holes 32 define a diameter that is between 15-45% of the diameter of the interior diameter of the tip 10. Such embodiments include those where the end/tip vent holes 32 define a diameter that is between 90-100% of the diameter of the circumferential vent holes 30 and the end/tip vent holes 32 define a diameter that is between 25-35% of the diameter of the interior diameter of the tip 10.

The above-described relative diameter sizes produce relative values for the areas proscribed by the vent holes 30, 32. In one embodiment, circumferential vent holes 30 define an area that is between 5-35% of the interior lumen cross-sectional area of the tip 10. More particularly, embodiments are envisioned where circumferential vent holes 30 define an area that is between 5-15% of the interior lumen of the tip 10. Similarly, embodiments are envisioned where the end (tip) vent holes 32 define an area that is between 5-10% of the area of the lumen of the tip 10. Further, embodiments are envisioned where the end/tip vent holes 32 define an area that is between 35-55% of the area of circumferential vent holes 30. More particularly, embodiments are envisioned where the end/tip vent holes 32 define an area that is between 30-50% of the area of circumferential vent holes 30.

Higher flow embodiments are envisioned where the end/tip vent holes 32 define an area that is between 80-100% of the area of the circumferential vent holes 30 and the end/tip vent holes 32 define an area that is between 5-35% of the area of the interior diameter of the tip 10. Such embodiments include those where the end/tip vent holes 32 define an area that is between 90-100% of the area of the circumferential vent holes 30 and the end/tip vent holes 32 define an area that is between 5-15% of the area of the interior of the tip 10.

The total area of the circumferential vent holes 30 (the sum of the areas of all the circumferential vent holes 30) is between 125-450% of the interior lumen cross-sectional area of the tip 10. This is inclusive of embodiments having a total area of the circumferential vent holes 30 that is between 130-150% of the interior lumen cross-sectional area of the tip 10. The total area of the end/tip vent holes 32 is between 3-10% of the total area of the circumferential vent holes 30. This is inclusive of embodiments where the total area of the end/tip vent holes 32 is between 6-8% of the total area of the circumferential vent holes 30. The total area of the end/tip vent holes 32 is between 5-20% of the area of the area of the interior of the tip 10. This is inclusive of embodiments where the total area of the end/tip vent holes 32 is between 8-12% of the area of the area of the interior of the tip 10.

In high flow embodiments, the end/tip vent holes 32 define a total area that is 14-18% of the total circumferential vent hole 30 area and define a total end/tip vent hole 32 area that is 20-70% of lumen area. This is inclusive of embodiments where the end/tip vent holes 32 define a total area that is 15.5-16.5% of the total circumferential vent hole 30 area and define a total end/tip vent hole 32 area that is 20-24% of lumen area.

The total area of the circumferential vent holes 30 (the sum of the areas of all of the circumferential vent holes 30) and all of the end/tip vent holes 32 is greater than 130% of the interior lumen cross-sectional area of the tip 10. This is inclusive of embodiments having a total area of the circumferential vent holes 30 and end/tip vent holes 32 that is greater than 145% of the interior lumen cross-sectional area of the tip 10.

Operationally, tip 10 provides a flat distal wall 18 that allows direct visualization of the operational field with a microscope, telescope, or with loupes. Stated differently, distal wall 18 does not extend radially beyond the circumferential wall 16. The distal wall 18 further provides a surface with some suction to cause adherence of matter thereto while providing that such suction is not so strong as to cause dissection or tearing of tissue. Accordingly, tip 10 can be used to “grab” and position material, such as grafts and then subsequently release such material through use of a common thumb-hole bypass valve. Still further, suction can be placed adjacent delicate tissue to remove liquid therefrom while having a reduced likelihood of adverse impact on the delicate tissue. Tip 10 further provides that encountering tissue having a size that is greater than the tip of suction wand 400 is less likely to provide a clog and/or a full clog of the suction pathway. If such “larger matter” is engaged by one or more vent holes 30, 32 it is less likely to also engage all the other vent holes 30, 32 and thereby not fully occlude all available suction paths.

Embodiments are envisioned where tip 10 is bendable to be able to access locations impossible or less convenient for statically shaped tips. Such bendable tips can have bellows such as a “bendy straw” and/or can be reinforced by a bendable metal to hold a position. Some embodiments are specifically non-magnetic so as to not interfere with tip navigation devices. Still other embodiments are formed specifically to be compatible with tip navigation devices.

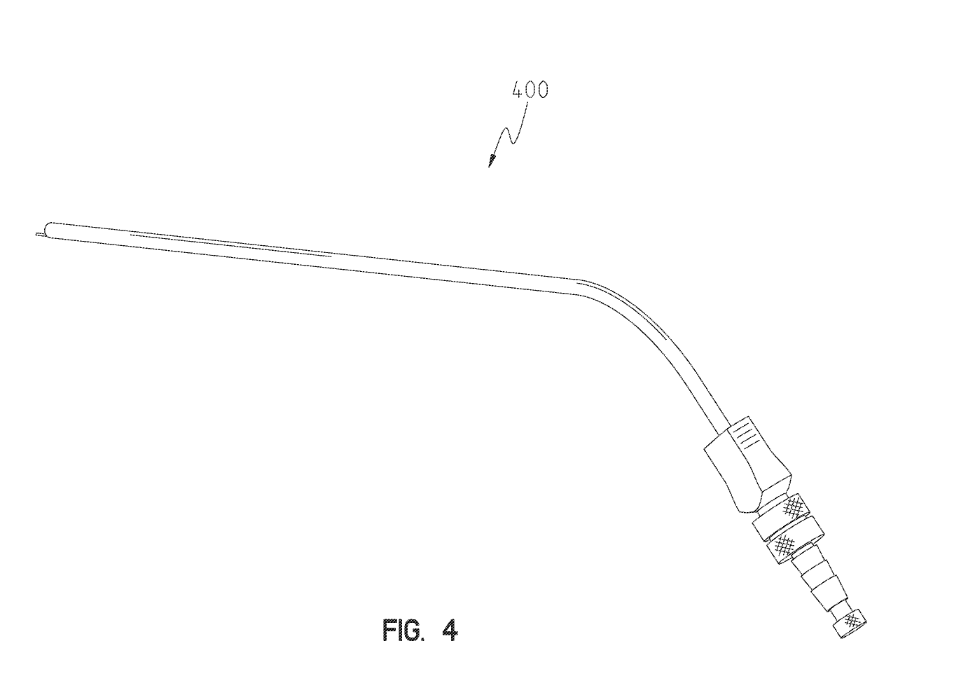

Embodiments are envisioned where an adapter is provided such that tip 10 can be placed on suction devices having a diameter larger than diameters typically seen in suction devices of the type shown in FIG. 4.

End/tip vent holes when in communication with a suction source, are able to provide for manipulation of matter (including delicate matter), such as grafts without allowing such matter to be received in the lumen of tip 10 (and suctioned away). In use, with the suction actively applied to tip 10, a user can abut distal wall 18 to the matter such that the matter is held against distal wall 18 by the suction force. The matter can then be placed as desired. A user can then cause a reduction in the suction force experienced by distal wall 18 to allow disengagement of the matter from distal wall 18. In one embodiment, such reduction in force is achieved by the removal of an occlusion over a relief port in suction wand 400. The disengagement of the matter from distal wall 18 provides that the matter is deposited at the desired location.

While the above embodiments of tip 10 described circumferential vent holes 30 as being substantially evenly distributed around the circumference, embodiments are envisioned where vent holes 32 are not uniformly distributed. Such non-uniform distribution includes embodiments where vent holes 32 are only on one side (distributed over 180-degrees of the 360-degree circumference). Such distributions can further allow for placement of the suction adjacent delicate tissue.

In addition to use with or as part of suction devices like that shown in FIG. 4, tips employing the teachings of this disclosure are also envisioned with Frazier (or other) surgical suction devices, tracheal suction catheters, and nasogastric/gastric suction devices, and other suction devices generally.

Furthermore, while the embodiments have been described as providing reduced trauma when used in areas of delicate tissue, tip 10 can also be used for more precise and delicate dissection of tissue.

The above detailed description and the examples described therein have been presented for the purposes of illustration and description only and not for limitation. For example, the operations described may be done in any suitable manner. The method may be done in any suitable order still providing the described operation and results. It is therefore contemplated that the present embodiments cover any and all modifications, variations or equivalents that fall within the spirit and scope of the basic underlying principles disclosed above and claimed herein.

What is claimed is:

1. A suction device including:
a side wall defining a lumen;
an open proximal end sized to provide coupling to a suction source;
a distal wall opposite the open proximal end, the distal wall at least partially occluding the lumen, the side wall extending between the proximal end and the distal wall;
a plurality of vent holes in communication with the lumen defined in the side wall; and
at least one vent hole in communication with the lumen defined in the distal wall.

2. The device of claim 1, wherein the side wall extends the full length of the device.

3. The device of claim 1, wherein the open proximal end defines an interior diameter that is equal to a diameter of the lumen.

4. The device of claim 1, wherein the distal wall is planar and perpendicular to a longitudinal axis of the device.

5. The device of claim 1, wherein the vent holes defined in the side wall are larger than the at least one vent hole defined in the distal wall.

6. The device of claim 1, wherein a total area of all of the at least one vent hole defined in the distal wall is between 5% and 20% of a cross sectional area of the lumen.

7. The device of claim 1, wherein a total area of all of the at least one vent hole defined in the distal wall is between 3% and 10% of a total area of all of the plurality of vent holes defined in the side wall.

8. The device of claim 1, wherein a diameter of each of the at least one vent holes defined in the distal wall is between 35% and 55% of a diameter of each of the vent holes defined in the side wall.

9. The device of claim 1, wherein a total area of all of the at least one vent hole defined in the distal wall and the plurality of vent holes defined in the side wall is greater than 130% of a cross-sectional area of the lumen.

10. A suction device, including:
a tube-shaped body forming a lumen, the body having an outer surface, a distal tip defining a surface having at least two openings therein, the at least two openings being in communication with the lumen, a proximal end having an opening in communication with the lumen, the proximal end shaped to couple to a suction source, and a distal end having a distal wall, the distal wall having at least one opening in communication with the lumen, the at least one opening having a diameter smaller than a diameter of the lumen.

11. The device of claim 10, wherein the lumen is sized to receive a suction source therein.

12. The device of claim 11, further including a stop disposed within the lumen that limits the travel of the suction source within the lumen.

13. The device of claim 10, wherein the tube-shaped body further includes a circumferential wall and including a plurality of circumferential wall vent holes.

14. The device of claim 13, wherein the plurality of circumferential wall vent holes are spaced unevenly around the tube shaped body.

15. The device of claim 14, wherein all of the circumferential wall vent holes are disposed within an 180-degree portion of the tube-shaped body such that at least 180-degrees of the tube-shaped body is free of circumferential wall vent holes.

16. The device of claim 13, wherein a total area of all of the at least one opening defined in the distal wall and all of the plurality of vent holes defined in the circumferential wall is greater than 130% of a cross-sectional area of the lumen.

17. The device of claim 13, wherein a diameter of each of the at least one opening defined in the distal wall is between 35% and 55% of the diameter of each of the vent holes defined in the circumferential wall.

18. The device of claim 13, wherein a total area of all the at least one opening defined in the planar distal wall is between 3% and 10% of a total area of all the plurality of vent holes defined in the circumferential wall.

19. The device of claim 10, wherein the distal wall is planar and perpendicular to a longitudinal axis of the device.

20. A method of manipulating matter in a surgical environment including:
obtaining a suction device including:
a side wall defining a lumen;
an open proximal end sized to provide coupling to a suction source;
a distal wall opposite the open proximal end, the distal wall at least partially occluding the lumen, the side wall extending between the proximal end and the distal wall;
a plurality of vent holes in communication with the lumen defined in the side wall; and
at least one vent hole in communication with the lumen defined in the distal wall;
placing the proximal end of the suction device in communication with a suction source;
engaging matter with the distal wall such that said matter adheres to the distal wall via suction supplied by the suction source without allowing said matter to enter the lumen;
placing the matter at a desired location; and
causing a reduction in suction provided at the distal wall to allow disengagement of the matter from the distal wall.
